(19) United States
(12) Patent Application Publication
(10) Pub. No.: US 2026/0198843 A1
(43) Pub. Date: Jul. 16, 2026

(54) PREDICTION OF LABOR ONSET USING IMPLANTABLE MEDICAL DEVICES

(71) Applicant: Medtronic, Inc., Minneapolis, MN (US)

(72) Inventors: Taya H. Cassens, Blaine, MN (US); Santhisagar Vaddiraju, Plymouth, MN (US); Veronica Ramos, Miami, FL (US); Kate L. Sherman, Maple Grove, MN (US)

(21) Appl. No.: 19/422,538

(22) Filed: Dec. 17, 2025

Related U.S. Application Data
(60) Provisional application No. 63/744,579, filed on Jan. 13, 2025.

(51) Int. Cl. A61B 5/00; A61B 5/0205; A61B 5/024; A61B 5/0245; G16H 50/30

(52) U.S. Cl. CPC A61B 5/4343; A61B 5/0205; A61B 5/6861; A61B 5/7267; A61B 5/7275; G16H 50/30; A61B 5/02405; A61B 5/02416; A61B 5/0245

(57) ABSTRACT

Systems and methods for monitoring a pregnant patient are disclosed herein. In some embodiments, a system includes a medical device configured to be implanted in a pregnant patient, processing circuitry, and a memory operably coupled to the processing circuitry. The medical device can include a sensor configured to obtain sensor data indicative of a heart rate of the pregnant patient over a first time period. The memory can store instructions that, when executed by the processing circuitry, cause the system to perform operations including receiving the sensor data, determining a heart rate variability (HRV) based on the sensor data, receiving time index data indicative of a timing of the patient's pregnancy, generating a premature labor risk score based on the HRV data and the time index data, and outputting an indication of the premature labor risk score.

### RELATED APPLICATIONS

This application claims the benefit of U.S. Provisional Application Ser. No. 63/744,579, filed Jan. 13, 2025, the entire contents of each of which are incorporated herein by reference.

### TECHNICAL FIELD

The present technology generally relates to medical devices, and in particular, to prediction of labor onset using implantable medical devices.

### BACKGROUND

Preterm birth and/or low birthweight are some of the leading causes of infant mortality in the United States. Babies who go on to survive preterm births may develop various health issues, including breathing problems, feeding difficulties, cerebral palsy, developmental delay, vision problems, and hearing problems. Furthermore, women who undergo premature labor are at significantly increased risk of future cardiovascular disease. Several factors may affect the likelihood that a woman will experience premature labor, including demographic characteristics, medical conditions, social characteristics, and behavioral characteristics. Early detection and/or prediction of premature labor can enable significant improvements in outcomes through interventional therapies and/or treatments such as steroid injections.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a conceptual diagram illustrating an example medical system configured to monitor a pregnant patient, in accordance with embodiments of the present technology.

FIG. 2A is a conceptual diagram of an example of an implantable medical device for monitoring a patient, in accordance with embodiments of the present technology.

FIG. 2B is a functional schematic diagram of an implantable medical device, in accordance with the present technology.

FIG. 3 is a conceptual perspective schematic diagram of an implantable medical device, in accordance with embodiments of the present technology.

FIG. 4 is a graph illustrating an example relationship between heart rate variability and time until delivery, in accordance with embodiments of the present technology.

FIG. 5 is a flow diagram illustrating a method for predicting premature labor onset in a pregnant patient, in accordance with embodiments of the present technology.

FIG. 6 is a block diagram illustrating an algorithm architecture for generating a premature labor risk score, in accordance with embodiments of the present technology.

FIG. 7 is a block diagram illustrating an algorithm architecture for determining whether a patient is at high risk of premature labor, in accordance with embodiments of the present technology.

### DETAILED DESCRIPTION

The present technology relates to systems, devices, and methods for monitoring a pregnant patient, e.g., to predict labor onset. In some embodiments, for example, a system provided herein includes a medical device configured to be implanted in a pregnant patient, where the medical device includes a sensor for obtaining sensor data indicative of a heart rate of the pregnant patient over a first time period. The medical device can be an implantable cardiac monitor including electrodes configured to obtain electrophysiological data of the patient, such as an electrocardiogram. The system can also include processing circuitry and a memory storing instructions that, when executed by the processing circuitry, cause the system to perform operations for receiving and analyzing sensor data to determine a premature labor risk score. In some embodiments, the operations include receiving sensor data indicative of the heart rate of the pregnant patient over the first time period from the sensor, and determining heart rate variability (HRV) data for the pregnant patient over the first time period based on the sensor data. The system can also receive time index data indicative of a timing of the patient's pregnancy. Subsequently, the system can generate a premature labor risk score based on the HRV data and the time index data, where the premature labor risk score represents a likelihood that the pregnant patient will experience premature labor onset during a second time period subsequent to the first time period. In some embodiments, the premature labor risk score is generated using a machine learning algorithm that is trained on HRV data and labor onset data from a plurality of patients. Optionally, the machine learning algorithm can consider other types of physiological characteristics (e.g., activity levels, sleep parameters, fluid levels, respiration rate, blood pressure, body temperature, biochemical species levels) in determining the likelihood of premature labor onset. The system can then output an indication of the premature labor risk score, where the indication is configured to be displayed to a user via a computing device.

The present technology can provide many advantages compared to conventional approaches to pregnancy monitoring. Implantable devices may have more proximate access to the patient's physiological signals and/or may require less patient interaction and/or maintenance compared to other types of medical devices, resulting in improved signal quality and/or patient compliance. In contrast, wearable devices may encounter more significant issues with skin color (e.g., if using a patch or other skin-mounted format), motion artifacts, and/or good electrode contact (e.g., if the wearable device is not properly secured). Furthermore, implantable devices may enable convenient, continuous monitoring of the patient during post-pregnancy, thereby allowing for continued surveillance for issues that may subsequently arise in the patient (e.g., cardiovascular disease).

The present technology can also provide advantages related to prediction of labor onset of the pregnant patient. For example, the determination of a premature labor risk score that represents a likelihood of a pregnant patient experiencing premature labor onset at a later time period as described herein can alert the user (e.g., clinician, patient) of the pregnant patient's current and/or future condition based on various physiological characteristics of the pregnant patient and/or the fetal patient. The ability to predict whether the pregnant patient will experience premature labor onset during a future time period may extend the timeline for the clinician to prescribe interventional treatments for the pregnant patient in order to improve outcomes for the pregnant patient and/or the fetal patient. In some embodiments, the systems, devices, and methods herein implement a machine learning algorithm that utilizes not only heart rate variability, but other physiological characteristics that may provide important insights relevant to prediction of labor onset and/or yield a more accurate prediction result.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Systems and Devices for Pregnancy Monitoring

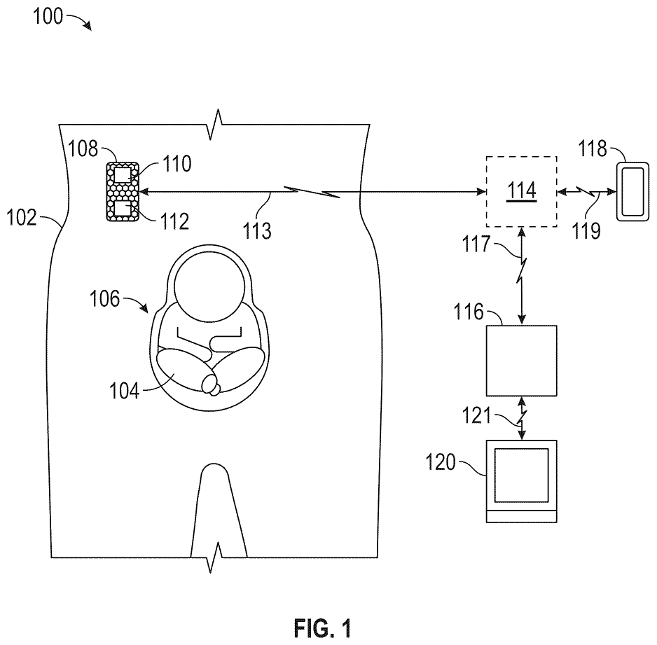

FIG. 1 is a conceptual diagram illustrating an example medical system 100 configured to monitor a pregnant patient 102, in accordance with embodiments of the present technology. In the illustrated example, the patient 102 is carrying a fetal patient 104 within a reproduction system 106 (e.g., a uterus). The system 100 may be configured to sense one or more physiological characteristics of the patient 102 and/or the fetal patient 104 in order to monitor and/or predict the likelihood of one or more obstetric conditions, such as premature labor onset (e.g., labor occurring before week 37 of the pregnancy and/or before an ideal due date for the patient 102), preeclampsia, chronic hypertension, gestational hypertension, sepsis, heart failure, cardiac arrhythmias, gestational diabetes, hemorrhage, postpartum depression, etc.

The system 100 includes one or more medical devices, such as a medical device 108. The medical device 108 may be an implantable medical device that is located internally within the patient's body. In some embodiments, the medical device 108 is implanted subcutaneously, e.g., entirely implanted subcutaneously. The medical device 108 may be implanted at any suitable location, such as a location near a heart, abdomen, placenta, uterus, or cranium of the patient 102. For example, the medical device 108 may be implanted in a lateral abdominal or inframammary region of patient 102.

In some embodiments, the medical device 108 is placed subcutaneously in the patient 102, e.g., outside of a thoracic cavity of the patient 102 in a pectoral location near or over the patient's heart. The medical device 108 may be positioned near the sternum near or just below the level of the heart, e.g., at least partially within the cardiac silhouette. For example, the medical device 108 can be placed in a subcutaneous pocket located over an intercostal space (e.g., over the 4th intercostal space), and positioned at a desirable angle and/or displacement relative to the patient's sternum (e.g., between about 0 and 45 degrees relative to the sternum, about 2 cm from the left edge of the sternum). Once inserted, the medical device 108 may go through suitable setup and/or calibration processes.

In some embodiments, the medical device 108 is a cardiac monitor, a defibrillator, a cardiac resynchronization pacer/defibrillator, a pacemaker, an implantable pressure sensor, a neurostimulator, or any other implantable medical device. For example, the medical device 108 may be an insertable cardiac monitor (ICM) (also referred to interchangeably herein as an “implantable cardiac monitor”), such as the Medtronic LINQ™ or LINQ II™ ICM, or other ICM similar to, e.g., a version or modification of the LINQ™ or LINQ II™ ICM. A representative example of an ICM is described in U.S. Pat. No. 10,413,207, the disclosure of which is incorporated herein by reference in its entirety.

The medical device 108 can be configured to sense one or more physiological characteristics of the patient 102 and/or the fetal patient 104. Examples of physiological characteristics include any of the following: heart rate (e.g., minimum heart rate, maximum rate, heart rate variability over time), blood pressure (e.g., systolic blood pressure, diastolic blood pressure), oxygen saturation level (e.g., an SpO2 indicative of blood oxygen saturation and/or an StO2 indicative of tissue oxygen saturation), respiration rate, fluid level (e.g., subcutaneous fluid level, amniotic fluid level), body temperature, activity level (e.g., resting, low activity, high activity), biochemical species level (e.g., blood glucose level, hormone level), and/or sleep parameters (e.g., total sleep per night, time spent in REM sleep per night, sleep position, night wakings).

In some embodiments, the medical device 108 is configured to sense physiological characteristics of the patient 102 only or of the fetal patient 104 only. Alternatively, the medical device 108 may be configured to sense physiological characteristics of both the patient 102 and the fetal patient 104 (e.g., concurrently or sequentially), and the system 100 may subsequently distinguish between the physiological characteristics of the patient 102 and the physiological characteristics of the fetal patient 104 (e.g., via blind source separation, wavelet methodologies). Optionally, the system 100 may include a first medical device 108 that senses physiological characteristics of the patient 102 only and a second medical device 108 that senses physiological characteristics of the fetal patient 104 only. In such embodiments, the first and second medical devices 108 may be the same type of medical device or may be different types of medical devices.

The medical device 108 may include (e.g., mechanically support) and/or otherwise be operably connected to one or more sensors 110 and/or sensing circuitry 112. The sensors 110 can be configured to generate sensor data indicative of one or more physiological characteristics of the patient 102 and/or the fetal patient 104. Examples of sensors 110 that may be used include electrical sensors (e.g., electrodes), optical sensors (e.g., photoplethysmography (PPG) sensors), motion sensors (e.g., accelerometers, gyroscopes, magnetometers, inertial measurement units (IMU)), temperature sensors (e.g., thermistors), impedance sensors, acoustic sensors (e.g., microphones), and biochemical sensors. Examples of sensor data that may be obtained using one or more sensors 110 include electrophysiological data (e.g., electrocardiogram (ECG) (also referred to as cardiac electrogram (EGM) data, echocardiogram data, electromyography data, impedance data), optical data (e.g., PPG data), motion data, posture data, temperature data, audio signal data, and biochemical signal data. The sensor data may be obtained over any suitable time period, such as a time period of at least 12 hours, 24 hours, 36 hours, 48 hours, 3 days, 4 days, 5 days, 6 days, 1 week, 2 weeks, 3 weeks, 4 weeks, 8 weeks, 10 weeks, 12 weeks, 15 weeks, 20 weeks, 30 weeks, or 40 weeks. In some embodiments, sensor data may be obtained at periodic intervals (e.g., continuously) during the time period, such as once every minute, 2 minutes, 5 minutes, 10 minutes, 15 minutes, 20 minutes, 30 minutes, every hour, 2 hours, 5 hours, 10 hours, 12 hours, 24 hours, 36 hours, or 48 hours.

The sensor data produced by the sensor 110 may be analyzed to determine the one or more physiological characteristics of the patient 102 and/or the fetal patient 104. In some embodiments, a physiological characteristic may be determined using a single type of sensor data (e.g., a heart rate based on ECG data, a blood pressure based on PPG data, an oxygen saturation level based on PPG data, a body temperature based on temperature data, a fluid level based on impedance data, etc.). In some embodiments, a physiological characteristic may be determined based on two or more different types of sensor data (e.g., a heart rate based on ECG data and PPG data; an activity level based on ECG data and motion data; a sleep parameter based on motion data and/or posture data; etc.).

In some embodiments, the sensing circuitry 112 is configured to communicate (e.g., via a link 113) the sensor data to processing circuitry 114. The link 113 may be a wired or wireless communication link, depending on the relative locations of the sensing circuitry 112 and the processing circuitry 114. Optionally, the sensing circuitry 112 may be omitted, e.g., in embodiments where the processing circuitry 114 is located onboard the medical device 108, as discussed further below.

The processing circuitry 114 can receive and analyze the sensor data produced by the sensors 110 of the medical device 108 to determine the physiological characteristics of the patient 102 and/or the fetal patient 104. The processing circuitry 114 may be included in (e.g., mechanically supported by) the medical device 108, by an external device 116 that is external to the patient's body (e.g., an external programmer for controlling the medical device 108), or a combination thereof. In some embodiments, the medical device 108 includes (e.g., mechanically supports) a first portion of the processing circuitry 114 and the external device 116 includes (e.g., mechanically supports) a second portion of the processing circuitry 114. In some embodiments, the processing circuitry 114 or a portion thereof may be included in (e.g., mechanically supported by) a patient input/output (IO) device 118 and/or a clinician IO device 120.

In some embodiments, the processing circuitry 114 uses the determined physiological characteristics to monitor a current condition of the patient 102 and/or the fetal patient 104. For example, the physiological characteristics can be determined for a first time period (e.g., using sensor data obtained during the first time period), and then used to determine the condition of the patient 102 and/or the fetal patient 104 during the first time period. The current condition of the patient 102 and/or the fetal patient 104 can be determined based on, for example, the value of a physiological characteristic (e.g., whether the average, median, minimum, and/or maximum value is within a normal range), the rate of change of a physiological characteristic, the variability of a physiological characteristic, and/or other relevant statistics or features.

In some embodiments, the processing circuitry 114 uses the determined physiological characteristics to predict a future condition of the patient 102 and/or the fetal patient 104. For example, the physiological characteristics can be determined for a first time period (e.g., using sensor data obtained during the first time period), and then used to predict the future condition of the patient 102 and/or the fetal patient 104 during a second time period subsequent to the first time period. The prediction can be based on, for example, the value of a physiological characteristic (e.g., whether the average, median, minimum, and/or maximum value is within a normal range), the rate of change of a physiological characteristic, the variability of a physiological characteristic, and/or other relevant statistics or features of one or more physiological characteristics measured during the first time period. The predicted future condition can be a prediction of whether the patient 102 and/or the fetal patient 104 are likely to experience a health event that places the health of the patient 102 and/or the fetal patient 104 at risk. For example, physiological characteristics of the patient 102 and/or the fetal patient 104 can be used to predict whether the patient 102 is likely to experience premature labor onset, e.g., as discussed in Section II below. Other conditions that may be predicted include preeclampsia, chronic hypertension, gestational hypertension, sepsis, heart failure, cardiac arrhythmias, gestational diabetes, hemorrhage, postpartum depression, etc.

In some embodiments, the processing circuitry 114 is configured to analyze the sensor data to determine a heart rate of the patient 102 and/or the fetal patient 104, such as a maximum heart rate, a minimum heart rate, and/or a heart rate variability. For example, the processing circuitry 114 may be configured to determine the heart rate using sensor data from the one or more sensors 110, such as an electrical sensor (e.g., an electrode), an optical sensor (e.g., a PPG sensor), etc. In some embodiments, the determined heart rate may be based on sensor data received over a period of time (e.g., a statistical parameter, a trend, or other attributes based on time-series data). The processing circuitry 114 may use the determined heart rate to determine and/or a predict a condition of the patient 102 and/or the fetal patient 104. For example, the processing circuitry 114 may compare the determined heart rate to a maximum heart rate associated with an obstetric condition (e.g., 100 BPM), a minimum heart rate associated with an obstetric condition (e.g., 60 BPM), etc. Optionally, the determined heart rate may be used in combination with one or more additional physiological characteristics to determine and/or a predict a condition of the patient 102 and/or the fetal patient 104.

In some embodiments, the processing circuitry 114 is configured to analyze the sensor data to determine a blood pressure of the patient 102 and/or the fetal patient 104, such as a systolic blood pressure and/or a diastolic blood pressure. For example, the processing circuitry 114 may be configured to determine the blood pressure using sensor data from the one or more sensors 110, such as an optical sensor (e.g., a PPG sensor), etc. In some embodiments, the determined and/or predicted blood pressure may be based on sensor data received over a period of time (e.g., a statistical parameter, a trend, or other attributes based on time-series data). The processing circuitry 114 may use the determined blood pressure to determine and/or a predict a condition of the patient 102 and/or the fetal patient 104. For example, the processing circuitry 114 may compare the determined blood pressure to a maximum blood pressure associated with an obstetric condition (e.g., a maximum systolic blood pressure of 130 mm Hg, a maximum diastolic blood pressure of 80 mm Hg), a minimum blood pressure associated with an obstetric condition (e.g., a minimum systolic blood pressure of 100 mm Hg, a minimum diastolic blood pressure of 60 mm Hg), etc. Optionally, the determined blood pressure may be used in combination with one or more additional physiological characteristics to determine and/or a predict a condition of the patient 102 and/or the fetal patient 104.

In some embodiments, the processing circuitry 114 is configured to analyze the sensor data to determine an oxygen saturation level of the patient 102 and/or the fetal patient 104. For example, the processing circuitry 114 may be configured to determine the oxygen saturation level using sensor data from the one or more sensors 110, such as an optical sensor (e.g., a PPG sensor), etc. In some embodiments, the determined oxygen saturation level may be based on sensor data received over a period of time (e.g., a statistical parameter, a trend, or other attributes based on time-series data). The processing circuitry 114 may use the determined oxygen saturation level to determine and/or a predict a condition of the patient 102 and/or the fetal patient 104. For example, the processing circuitry 114 may compare the determined oxygen saturation level to a maximum oxygen saturation level associated with an obstetric condition (e.g., 100%), a minimum oxygen saturation level associated with an obstetric condition (e.g., 95%), etc. Optionally, the determined oxygen saturation level may be used in combination with one or more additional physiological characteristics to determine and/or a predict a condition of the patient 102 and/or the fetal patient 104.

In some embodiments, the processing circuitry 114 is configured to analyze the sensor data to determine a respiration rate of the patient 102 and/or the fetal patient 104. For example, the processing circuitry 114 may be configured to determine the respiration rate using sensor data from the one or more sensors 110, such as an electrical sensor (e.g., an electrode), a motion sensor (e.g., an accelerometer), etc. In some embodiments, the determined respiration rate may be based on sensor data received over a period of time (e.g., a statistical parameter, a trend, or other attributes based on time-series data). The processing circuitry 114 may use the determined respiration rate to determine and/or a predict a condition of the patient 102 and/or the fetal patient 104. For example, the processing circuitry 114 may compare the determined respiration rate to a maximum respiration rate associated with an obstetric condition (e.g., 16 breaths per minute for the patient 102, 70 breaths per minute for the fetal patient 104), a minimum respiration rate associated with an obstetric condition (e.g., 12 breaths per minute for the patient 102, 30 breaths per minute for the fetal patient 104), etc. Optionally, the determined respiration rate may be used in combination with one or more additional physiological characteristics to determine and/or a predict a condition of the patient 102 and/or the fetal patient 104.

In some embodiments, the processing circuitry 114 is configured to analyze the sensor data to determine a body temperature of the patient 102 and/or the fetal patient 104. For example, the processing circuitry 114 may be configured to determine the body temperature using sensor data from the one or more sensors 110, such as a temperature sensor (e.g., a thermistor), etc. In some embodiments, the determined body temperature may be based on sensor data received over a period of time (e.g., a statistical parameter, a trend, or other attributes based on time-series data). The processing circuitry 114 may use the determined body temperature to determine and/or a predict a condition of the patient 102 and/or the fetal patient 104. For example, the processing circuitry 114 may compare the determined and/or predicted body temperature to a maximum body temperature associated with an obstetric condition (e.g., 99.3 degrees F.), a minimum body temperature associated with an obstetric condition (e.g., 97.9 degrees F.), etc. Optionally, the determined body temperature may be used in combination with one or more additional physiological characteristics to determine and/or a predict a condition of the patient 102 and/or the fetal patient 104.

In some embodiments, the processing circuitry 114 is configured to analyze the sensor data to determine an activity level of the patient 102 and/or the fetal patient 104, such as whether the patient is resting, engaging in low-effort activities (e.g., walking), or engaging in high-effort activities (e.g., running). For example, the processing circuitry 114 may be configured to determine the activity level using sensor data from the one or more sensors 110, such as a motion sensor (e.g., accelerometer), etc. In some embodiments, the determined activity level may be based on sensor data received over a period of time (e.g., a statistical parameter, a trend, or other attributes based on time-series data). The processing circuitry 114 may use the determined activity level to determine and/or a predict a condition of the patient 102 and/or the fetal patient 104. For example, the processing circuitry 114 may compare the determined activity level to a maximum activity level associated with an obstetric condition, a minimum activity level associated with an obstetric condition, etc. Optionally, the determined activity level may be used in combination with one or more additional physiological characteristics to determine and/or a predict a condition of the patient 102 and/or the fetal patient 104.

In some embodiments, the processing circuitry 114 is configured to analyze the sensor data to determine a sleep parameter of the patient 102, such as the total amount of sleep per night, the time spent in various sleep stages (e.g., REM sleep), etc. For example, the processing circuitry 114 may be configured to determine the sleep parameter using sensor data from the one or more sensors 110, such as a motion and/or posture sensor (e.g., accelerometer), etc. In some embodiments, the determined sleep parameter may be based on sensor data received over a period of time (e.g., a statistical parameter, a trend, or other attributes based on time-series data). The processing circuitry 114 may use the determined sleep parameter to determine and/or a predict a condition of the patient 102 and/or the fetal patient 104. For example, the processing circuitry 114 may compare the determined and/or predicted sleep parameter to a maximum sleep parameter associated with an obstetric condition, a minimum sleep parameter associated with an obstetric condition, etc. Optionally, the determined sleep parameter may be used in combination with one or more additional physiological characteristics to determine and/or a predict a condition of the patient 102 and/or the fetal patient 104.

In some embodiments, the processing circuitry 114 is configured to analyze the sensor data to determine a fluid level of the patient 102, such as a subcutaneous fluid level, an amniotic fluid level, etc. For example, the processing circuitry 114 may be configured to determine the fluid level using sensor data from the one or more sensors 110, such as an impedance sensor, etc. In some embodiments, the determined fluid level may be based on sensor data received over a period of time (e.g., a statistical parameter, a trend, or other attributes based on time-series data). The processing circuitry 114 may use the determined fluid level to determine and/or predict a condition of the patient 102 and/or the fetal patient 104. For example, the processing circuitry 114 may compare the determined fluid level to a maximum fluid level associated with an obstetric condition, a minimum fluid level associated with an obstetric condition, etc. Optionally, the determined fluid level may be used in combination with one or more additional physiological characteristics to determine and/or a predict a condition of the patient 102 and/or the fetal patient 104.

In some embodiments, the processing circuitry 114 is configured to analyze the sensor data to determine a glucose level of the patient 102. For example, the processing circuitry 114 may be configured to determine the glucose level using sensor data from the one or more sensors 110, such as a glucose sensor, etc. In some embodiments, the determined glucose level may be based on sensor data received over a period of time (e.g., a statistical parameter, a trend, or other attributes based on time-series data). The processing circuitry 114 may use the determined glucose level to determine and/or a predict a condition of the patient 102 and/or the fetal patient 104. For example, the processing circuitry 114 may compare the determined glucose level to a maximum glucose level associated with an obstetric condition, a minimum glucose level associated with an obstetric condition, etc. Optionally, the determined glucose level may be used in combination with one or more additional physiological characteristics to determine and/or predict a condition of the patient 102 and/or the fetal patient 104.

In some embodiments, the processing circuitry 114 is configured to analyze the sensor data to determine a hormone level within the patient 102, such as a level of luteinizing hormone, follicle-stimulating hormone, human chorionic gonadotropin hormone, estrogen, progesterone, or a combination thereof. For example, the processing circuitry 114 may be configured to determine the hormone level using sensor data from the one or more sensors 110, such as a biochemical sensor, a sensor configured to detect a heart rate of the patient 102 (e.g., an ECG), a urine sampling device, a weight scale, a blood pressure cuff, a temperature sensor, etc. In some embodiments, the determined and/or predicted hormone levels may be based on sensor data received over a period of time (e.g., a statistical parameter, a trend, or other attributes based on time-series data). The processing circuitry 114 may use the determined hormone level to determine and/or a predict a condition of the patient 102 and/or the fetal patient 104. For example, the processing circuitry 114 may compare the determined and/or predicted hormone level to a maximum hormone level associated with an obstetric condition, a minimum hormone level associated with an obstetric condition, etc. Optionally, the determined hormone level may be used in combination with one or more additional physiological characteristics to determine and/or a predict a condition of the patient 102 and/or the fetal patient 104.

Additional details and examples of techniques for monitoring and/or predicting a condition of the patient 102 and/or the fetal patient 104 are described in Section II below and also provided in U.S. Patent Publication No. 2023/0293023 and U.S. patent application Ser. No. 18/781,267, the disclosures of which are incorporated by reference herein in their entirety.

The processing circuitry 114 may be configured to communicate with another device, such as the external device 116 (e.g., via a link 117), the patient IO device 118 (e.g., via a link 119), and/or the clinician IO device 120 (e.g., via a link 121 or another communication link). The patient IO device 118, the external device 116, and/or the clinician IO device 120 may take the form of personal computing devices of the patient 102 and/or a clinician, such as a smartphone, smartwatch, or other smart apparel of the patient 102 or the clinician. The patient IO device 118, the external device 116, and/or the clinician IO device 120 may be any computing device configured for wireless communication with the processing circuitry 114, such as a desktop, laptop, or tablet computer, a smart home controller, alarm, thermostat, speaker, or other smart appliance, or any Internet of Things (IoT) device. The patient IO device 118, the external device 116, and/or the clinician IO device 120 may be configured to communicate with the processing circuitry 114 and/or with each other according to the Bluetooth® or Bluetooth® Low Energy (BLE and/or BTLE) protocols, as examples.

Any suitable information may be transmitted by the processing circuitry 114 to the other device, such as the sensor data obtained by the sensors 110, the physiological characteristics determined based on the sensor data, and/or an indication of a current and/or predicted future condition of the patient 102 and/or the fetal patient 104. The information can be displayed to a user (e.g., the patient 102 and/or the clinician) so that the patient 102 and/or the clinician may remain apprised of the likelihood of a given obstetric condition for the patient 102. In some embodiments, the processing circuitry 114 is configured to communicate first information to the patient IO device 118 and second information to the clinician IO device 120 such that, for example, the patient IO device 118 displays information useful to the patient 102 while the clinician IO device 120 displays additional and/or different information which might be useful to the clinician. The information received by the patient IO device 118, the external device 116, and/or the clinician IO device 120 can be displayed to the user in any suitable output format, such as a visual output, audio output, haptic output, or some other output which may be sensed by one or more of the senses of a human being.

In addition to or instead of transmitting information to another device, the processing circuitry 114 may be configured to record and/or store the information (e.g., sensor data, physiological characteristics, current and/or predicted future condition) to enable a user (e.g., the clinician and/or the patient 102) to review a history over some portion of or substantially the entirety of a time interval. For example, the processing circuitry 114 may be configured to communicate stored information to the external device 116, the clinician IO device 120, and/or the patient IO device 118. The processing circuitry 114 may be configured to communicate the stored information on a scheduled basis (e.g., twice daily, or on some other schedule) and/or in response to requests for information, such that the history remains substantially updated.

The system 100 illustrated in FIG. 1 can be modified in many ways. For example, although FIG. 1 illustrates a single medical device 108, the system 100 may optionally include multiple medical devices 108 (e.g., two, three, four, five, or more medical devices 108). For example, the system 100 may include a first medical device 108 that is implanted internally within the patient's body at a first location, and a second medical device 108 that is implanted internally within the patient's body at a second, different location. As another example, the system 100 may include a first medical device 108 that is implanted internally within the patient's body, and a second medical device 108 that is located external to the patient's body (e.g., a wearable device). In embodiments where the system 100 includes multiple medical devices 108, some or all of the medical devices 108 may include the same types of sensors 110, and/or some or all of the medical devices 108 may include different types of sensors 110.

Moreover, some of the components of the system 100 illustrated in FIG. 1 may be omitted, such as the external device 116, the patient IO device 118, and/or the clinician IO device 120. Alternatively or in combination, the system 100 may include additional components not shown in FIG. 1, such as additional external devices 116, additional patient IO devices 118, and/or additional clinician IO devices 120. Any of the components of the system 100 depicted as being separate can be combined with each other (e.g., the processing circuitry 114 may be incorporated into the medical device 108, the external device 116, the patient IO device 118, and/or the clinician IO device 120). Additionally, any of the components of the system 100 may be subdivided into smaller discrete subcomponents.

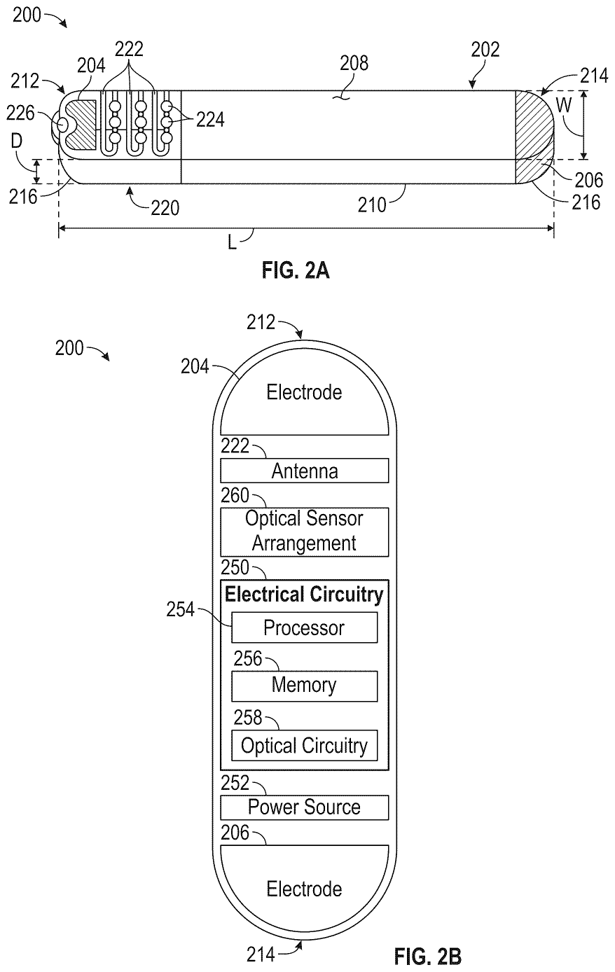

FIG. 2A is a conceptual diagram of an example of an implantable medical device (IMD) 200 (also referred to herein as a “cardiac monitoring device”) for monitoring a patient, in accordance with embodiments of the present technology. The implantable medical device 200 is an example of a medical device 108 that may be used in the system 100 of FIG. 1. In the example shown in FIG. 2A, the implantable medical device 200 may be embodied as a monitoring device having housing 202, a first (e.g., proximal) electrode 204, and a second (e.g., distal) electrode 206. The housing 202 may further comprise a first major surface 208, a second major surface 210, a first (e.g., proximal) end 212, and a second (e.g., distal) end 214. The housing 202 encloses electrical circuitry 250 and power source 252 (shown in FIG. 2B) located inside the implantable medical device 200 and protects the circuitry contained therein from body fluids. Electrical feedthroughs provide electrical connection of the electrodes 204 and 206.

In some embodiments such as that shown in FIG. 2A, the implantable medical device 200 is defined by a length L, a width W, and a thickness or depth D. The implantable medical device 200 may be in the form of an elongated rectangular prism wherein the length L is much larger than the width W, which in turn is larger than the depth D. In some embodiments, the geometry of the implantable medical device 200 (for example, a width W greater than the depth D) may be selected to allow the implantable medical device 200 to be inserted under the skin of the patient using a minimally invasive procedure and to remain in the desired orientation during insert. For example, the device shown in FIG. 2A may include radial asymmetries (notably, the rectangular shape) along the longitudinal axis that maintains the device in the proper orientation following insertion. For example, in some embodiments the spacing between the proximal electrode 204 and the distal electrode 206 may range from 30 millimeters (mm) to 55 mm, 35 mm to 55 mm, and from 40 mm to 55 mm and may be any range or individual spacing from 25 mm to 60 mm. In addition, the length L of the implantable medical device 200 may range from 30 mm to about 70 mm. In other embodiments, the length L may range from 40 mm to 60 mm, 45 mm to 60 mm and may be any length or range of lengths between about 30 mm and about 70 mm. In addition, the width W of the first major surface 208 may range from 3 mm to 10 mm and may be any single or range of widths between 3 mm and 10 mm. In some embodiments, the thickness or depth D of the implantable medical device 200 may range from 2 mm to 9 mm. For example, the depth D of the insertable cardiac monitor 200 may range from 2 mm to 5 mm and may be any single or range of depths from 2 mm to 9 mm. In addition, the implantable medical device 200, according to an example embodiment, has a geometry and size designed for ease of implant and patient comfort. Embodiments of the implantable medical device 200 described in this disclosure may have a volume of three cubic centimeters (cm) or less, 1.5 cubic cm or less or any volume between three and 1.5 cubic cm.

In the example shown in FIG. 2A, once inserted within the patient, the first major surface 208 faces outward, toward the skin of the patient while the second major surface 210 is located opposite the first major surface 208. In addition, in the example shown in FIG. 2A, the proximal end 212 and the distal end 214 are rounded to reduce discomfort and irritation to surrounding tissue once inserted under the skin of the patient. The implantable medical device 200, including instruments and methods for inserting the device 200, is described, for example, in U.S. Patent Publication No. 2014/0276928, incorporated herein by reference in its entirety.

In some embodiments, the proximal electrode 204 and the distal electrode 206 are used to sense cardiac signals for determining a cardiac event (e.g., bradycardia or asystole event) such as EGM signals, intra-thoracically or extra-thoracically, which may be sub-muscularly or subcutaneously. EGM signals may be stored in a memory of the implantable medical device 200, and EGM data may be transmitted via integrated antenna 222 to another medical device, which may be another implantable device or an external device.

In the example embodiment shown in FIG. 2A, the proximal electrode 204 is in close proximity to the proximal end 212 and the distal electrode 206 is in close proximity to the distal end 214. In this embodiment, the distal electrode 206 is not limited to a flattened, outward-facing surface, but may extend from the first major surface 208 around rounded edges 216 and onto the second major surface 210 so that the distal electrode 206 has a three-dimensional curved configuration. In the example embodiment shown in FIG. 2A, the proximal electrode 204 is located on the first major surface 208 and is substantially flat and outward facing. However, in other embodiments, the proximal electrode 204 may utilize the three-dimensional curved configuration similar to that of distal electrode 206, providing a three-dimensional proximal electrode (not shown in this embodiment). Additionally or alternatively, in other embodiments, the distal electrode 206 may utilize a substantially flat, outward-facing electrode located on the first major surface 208 similar to that shown with respect to the proximal electrode 204. The various electrode configurations allow for configurations in which the proximal electrode 204 and the distal electrode 206 are located on both the first major surface 208 and the second major surface 210. In other configurations, such as that shown in FIG. 2A, only one of the proximal electrode 204 and the distal electrode 206 is located on both the major surfaces 208 and 210. In still other configurations, both the proximal electrode 204 and the distal electrode 206 are located on one of the first major surface 208 or the second major surface 210 (e.g., the proximal electrode 204 located on the first major surface 208 while the distal electrode 206 is located on the second major surface 210). In some embodiments, the implantable medical device 200 may include electrodes on both the major surface 208 and the major surface 210 at or near the proximal and distal ends of the device, such that a total of at least four electrodes are included on the implantable medical device 200. The electrodes 204 and 206 may be formed of a plurality of different types of biocompatible conductive material (e.g. stainless steel, titanium, platinum, iridium, or alloys thereof), and/or may utilize one or more coatings such as titanium nitride or fractal titanium nitride.

In the example shown in FIG. 2A, the proximal end 212 includes a header assembly 220 that includes one or more of the proximal electrode 204, the integrated antenna 222, anti-migration projections 224, and/or suture hole 326. The integrated antenna 222 may be located on the same major surface (e.g., the first major surface 208) as the proximal electrode 204 and may also be included as part of the header assembly 220. The integrated antenna 222 allows the implantable medical device 200 to transmit and/or receive data. In some embodiments, the integrated antenna 222 may be formed on the opposite major surface as the proximal electrode 204, or may be incorporated within the housing 222 of the implantable medical device 200. In the example embodiment shown in FIG. 2A, the anti-migration projections 224 are located adjacent to the integrated antenna 222 and protrude away from the first major surface 208 to prevent longitudinal movement of the device 200, though may be arranged on any suitable surface of the implantable medical device 200. In the example embodiment shown in FIG. 2A, the anti-migration projections 224 include a plurality (e.g., nine) of small bumps or protrusions extending away from the first major surface 208; however, the anti-migration projections 224 may additionally or alternatively be located on the opposite major surface as the proximal electrode 204 and/or the integrated antenna 222. As shown in FIG. 2A, the suture hole 226, which may be used to help secure the implantable medical device 200 in the patient to prevent movement following insertion of the implantable medical device 200, may be located adjacent to the proximal electrode 204, though one or more suture holes 226 may additionally or alternatively be located in any other suitable location. In some embodiments, the header assembly 220 is a molded header assembly made from a polymeric or plastic material, which may be integrated or separable from the main portion of the implantable medical device 200.

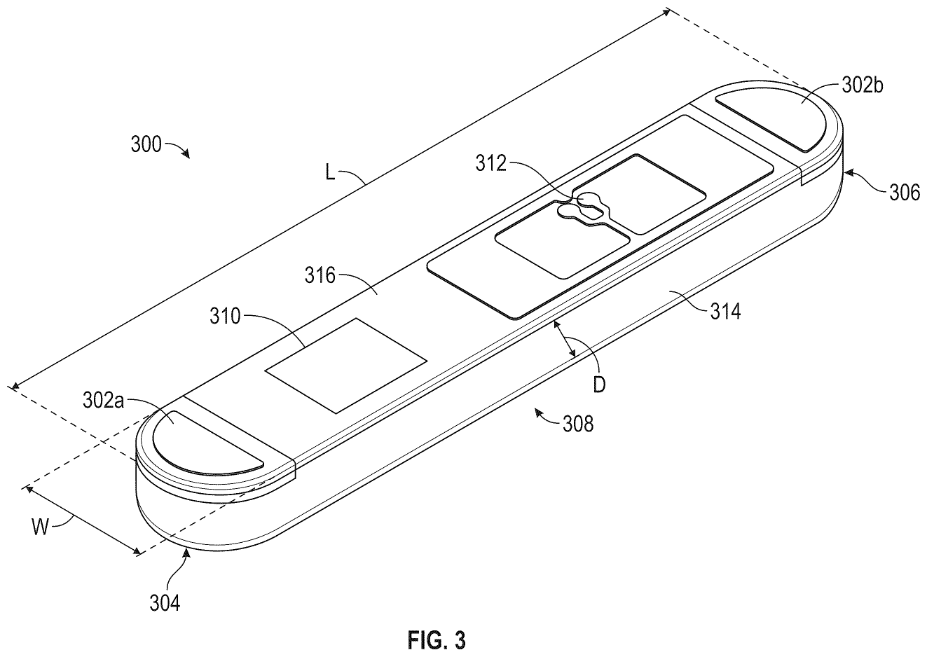

FIG. 2B is a functional schematic diagram of an implantable medical device, such as the implantable medical device 200 as shown in FIG. 2A, in accordance with embodiments of the present technology. Although the reference numbers refer to the implantable medical device 200, it should be understood that other implantable medical devices described herein (e.g., the implantable medical device 300 of FIG. 3) can include one or more components similar to that described below. The implantable medical device 200 includes housing 202, proximal electrode 204 located at proximal end 212, distal electrode 206 located at distal end 214, integrated antenna 222, electrical circuitry 250, and power source 252. In some embodiments, the implantable medical device 200 includes an optical sensor arrangement 260 comprising an emitter set of one or more optical light emitters and a detector set of one or more optical light detectors. The optical sensor arrangement 260 may, for example, be configured to provide a photoplethysmography (PPG) signal using the emitter and detector sets.

The optical sensor arrangement 260 can be configured to sense through one or more surfaces of the implantable medical device 200 (e.g., first major surface 208, second major surface 210, a surface of the header assembly 220). In some embodiments, one or more portions of the one or more surfaces comprise a material that is optically transparent to at least some wavelengths of light. For example, the one or more portions of the one or more surfaces through which the optical sensor arrangement 260 senses can be transparent to a red wavelength, transparent to a green wavelength, and/or transparent to an infrared wavelength. In some embodiments, the one or more portions of the one or more surfaces are optically transparent to visible light (e.g., electromagnetic radiation with a wavelength from approximately 380 nm to approximately 780 nm). The orientation of the optical sensor arrangement 260 with respect to the patient is based on the implantation of the implantable medical device 200. For example, in some embodiments (e.g., embodiments wherein the optical sensor arrangement 260 senses through the first major surface 208), the optical sensor arrangement 260 is directed away from a center of the patient (e.g., oriented distally) when the implantable medical device 200 is implanted within the patient, and thus is exposed to a maximal amount of ambient light.

Fidelity of ambient light sensing can correlate to factors external to the optical sensor arrangement 260, such as physical activity of a patient and the environment surrounding the patient. Sensing from the optical sensor arrangement 260 can be affected when the patient moves vigorously and/or when then patient is in an environment of intense and/or rapidly varying ambient light. In some embodiments, the optical sensor arrangement 260 is configured to sense in response to sensor data (e.g., motion data, optical data). For example, in some embodiments, a motion sensor (e.g., an accelerometer) senses physical activity of the patient and while the patient is below a first motion threshold as sensed by the motion sensor (e.g., when the patient is resting, when the patient remains still), electrical circuitry 250 directs (e.g., via optical circuitry 258) the optical sensor arrangement 260 to sense. In some embodiments, the motion sensor senses the patient is above a second motion threshold (e.g., the patient is moving vigorously) and the electrical circuitry 250 directs the optical sensor arrangement 260 not to sense. In some embodiments, the optical sensor arrangement 260 senses for a first period of time to determine whether optical data is suitable for the optical sensor arrangement 260 to sense at a second period of time (e.g., immediately after, continuously until another condition is met) or whether the optical sensor arrangement 260 should sense at a third period of time (e.g., after a duration of time such as 1 minute).

The electrical circuitry 250 may be coupled to the optical sensor arrangement 260 to sense optical signals (e.g., via the optical circuitry 258) corresponding to PPG and/or ambient light. The electrical circuitry 250 may be coupled to the proximal electrode 204 and the distal electrode 206 to sense cardiac signals and monitor events (e.g., arrythmia, etc.). The electrical circuitry 250 is also connected to transmit and receive communications via the integrated antenna 222. The power source 252 provides power to the electrical circuitry 250, as well as to any other components that require power. The power source 252 may include one or more energy storage devices, such as one or more rechargeable or non-rechargeable batteries. The implantable medical device 200 as shown in FIGS. 2A and 2B may be a monitoring-only device. However, in other examples, implantable medical device 200 may further provide therapy delivery capabilities.

The electrical circuitry 250 is configured to receive multiple signal types. For example, the electrical circuitry 250 can receive raw EGM signals monitored by the proximal electrode 204 and the distal electrode 206 and/or PPG signals monitored by the optical sensor arrangement 260. The electrical circuitry 250 may also include components/modules for converting a raw signal (e.g., EGM, PPG) to a processed signal that can be analyzed to detect sense events. Although not shown, the electrical circuitry 250 may include any discrete and/or integrated electronic circuit components that implement analog and/or digital circuits capable of producing the functions described for analyzing EGM and/or PPG signals to detect/verify bradycardia and/or asystole events. For example, the electrical circuitry 250 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, one or more processors 254 (shared, dedicated, or group) that executes one or more software or firmware programs, memory devices 256, or any other suitable components or combination thereof that provide the described functionality.

In some embodiments, the electrical circuitry 250 may include a sensing unit for monitoring signals detected (e.g., by the proximal electrode 204 and the distal electrode 206, by the optical sensor arrangement 260), and at least one sensing channel that utilizes an algorithm for identifying events in the signal (e.g., the EGM signal, the PPG signal). For example, sensed events (e.g., R-waves) are utilized to detect one or more cardiac episodes. In some embodiments, the electrical circuitry 250 includes the one or more processors 254 configured to receive information regarding the sensed events and implements one or more algorithms for determining whether a particular one or more events have occurred. In addition, the analog voltage signals received from the electrodes 204 and 206 and/or the optical sensor arrangement 260 may be passed to analog-to-digital (A/D) converters (ADC) included in the electrical circuitry 250, and stored in the memory unit 256 included as part of the electrical circuitry 250 for subsequent analysis with firmware executed by the processor(s) 254 included as part of the electrical circuitry 250.

The electrical circuitry 250 may control functions of the implantable medical device 200 and process signals received from the electrodes 204 and 206 (e.g., EGM signals) and/or the optical sensor arrangement 260 (e.g., optical signals) according to programmed signal analysis routines or algorithms. The implantable medical device 200 may include the optical circuitry 258 to facilitate optical signal detection, processing, and/or control. The implantable medical device 200 may include other optional sensors (not shown) for monitoring physiological signals, such as an activity sensor, pressure sensor, oxygen sensor, accelerometer, and/or other sensor used to monitor a patient. These may also be provided to the electrical circuitry 250 for processing.

The electrical circuitry 250 may similarly control monitoring time intervals and sampling rates according to a particular clinical application. In addition, electrical circuitry may include state machines or other sequential logic circuitry to control device functions and need not be implemented exclusively as a microprocessor.

The electrical circuitry 250 communicates with the integrated antenna 222 (shown in FIG. 3A) or other communication to transmit electrical signal data, e.g. EGM signal data, stored in memory or received from the electrical circuitry 250 in real time. The antenna 222 may be configured to transmit and receive communication signals via inductive coupling, electromagnetic coupling, tissue conductance, Near Field Communication (NFC), Radio Frequency Identification (RFID), BLUETOOTH®, WiFi, or other proprietary or non-proprietary wireless telemetry communication schemes.

The electrical circuitry 250 may include a communication module including the integrated antenna 222, so as to enable the implantable medical device 200 to communicate with one or more external devices located external to the device 200.

FIG. 3 is a conceptual perspective schematic diagram of an implantable medical device (IMD) 300, in accordance with embodiments of the present technology. The IMD 300 may be a leadless, subcutaneously implantable monitoring device including a proximal electrode 302a located at proximal end 304, a distal electrode 302b located at distal end 306 (collectively “electrodes 302”), a housing 308, electrical circuitry (not shown), an optical sensor arrangement 310 (comprising, for example optical sensor(s)), an integrated antenna 312, and a power source (not shown). In particular, the electrical circuitry is coupled to proximal electrode 302a and distal electrode 302b to sense cardiac signals and monitor events. The electrical circuitry may also be connected to transmit and receive communications via the integrated antenna 312. The power source can provide power to electrical circuitry, as well as to any other components that require power. The power source may include one or more energy storage devices, such as one or more rechargeable or non-rechargeable batteries. In some examples, the electrical circuitry includes processing circuitry and a storage device, such as memory, the memory being operatively coupled to the processing circuitry and configured to store data and/or instructions.

In the example shown in FIG. 3, the electrical circuitry may receive raw EGM or EMG (electromyography) signals monitored by the proximal electrode 302a and distal electrode 302b and raw optical signals monitored by the optical sensor arrangement 310. The electrical circuitry may include components/modules for converting the raw EGM signal to a processed EGM signal that can be analyzed to detect sense events and for converting the raw optical signals to calibrated processed optical signal(s) that can be analyzed to detect sense events. Although not shown, the electrical circuitry may include any discrete and/or integrated electronic circuit components that implement analog and/or digital circuits capable of producing the functions described for analyzing optical signal(s) to determine a health condition status of a patient. For example, the electrical circuitry may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, the electrical circuitry includes a sensing unit for monitoring the EGM signal detected by the respective proximal electrode 302a and the distal electrode 302b, as well as light signals received by the optical sensor arrangement 310, respectively. In one example, the electrical circuitry includes processing circuitry that is utilized to receive information regarding sensed events and to implement one or more algorithms for determining a health condition status of a patient. In addition, the analog voltage signals received from the electrodes 302 may be passed to analog-to-digital (A/D) converters included in the electrical circuitry and stored in the memory unit included as part of the electrical circuitry for subsequent analysis with firmware executed by the processor included as part of the electrical circuitry.

Some embodiments of the IMD 300 include a container 314 and an insulative cover 316. In some examples, the insulative cover 316 may include an optical window. In some examples, the optical window may be formed of the same material as insulative cover 316. In some examples, the optical window may be a portion of insulative cover 316. The proximal electrode 302a and the distal electrode 302b may be formed or placed on an outer surface of the cover 316. The electrical circuitry may be formed or placed on an inner surface of the cover 316, or within the container 314. In some examples, the antenna 312 is formed or placed on the inner surface of the cover 316. In other examples, the antenna 312 is formed or placed on the outer surface of the cover 316, or the antenna 312 may be formed or placed at least partially on the inner surface and partially on the outer surface of the cover 316. In some examples, the insulative cover 316 may be positioned over the open container 314 such that the container 314 and the cover 316 form the housing 308 and enclose the electrical circuitry (and in some cases the antenna 312) to protect the circuitries from fluids such as body fluids. For example, the housing 308 may be a hermetically-sealed housing configured for subcutaneous implantation within a patient, wherein at least the power source, the memory, and the processing circuitry are within the hermetically-sealed case, and in some examples, the optical sensor arrangement 310 is also within the hermetically-sealed case.

The electrical circuitry may be formed on the inner side of the insulative cover 316, such as by using flip-chip or wire bond integrated circuit packaging technology. The insulative cover 316 may be flipped onto the container 314. When flipped and placed onto the container 314, the components of IMD 300 formed on the inner side of the insulative cover 316 may be positioned in a gap defined by the container 314. The electrodes 302 and the antenna 312 (when placed or formed on the outer surface of the cover 316) may be electrically connected to sensing circuitry and communication circuitry, respectively, e.g., through one or more vias formed through the insulative cover 316. The insulative cover 316 may be formed of sapphire (i.e., corundum), glass, and/or any other suitable insulating material. The container 314 may be formed from any suitable material configured to house electrical circuitry, to support and mate with the cover 316 to isolate electrical circuitry from contact with tissue and/or fluids of a patient, and to be implantable within the patient. In some examples, the container 314 may house the power source (e.g., a battery). In some examples, the container 314 may also be electrically conductive. For example, the container 314 may be formed from titanium or any other suitable material (e.g., a biocompatible material). The electrodes 302 may be formed from any of stainless steel, titanium, platinum, iridium, or alloys thereof. In addition, the electrodes 302 may be coated with a material such as titanium nitride or fractal titanium nitride, although other suitable materials and coatings for such electrodes may be used.

In some embodiments, the IMD 300 is defined by a length L, a width W and thickness or depth D and is in the form of an elongated rectangular prism wherein the length L is much larger than the width W, which in turn is larger than the depth D, as illustrated in FIG. 3. In one example, the geometry of the IMD 300 (e.g., in particular the width W being greater than the depth D) is selected to allow the IMD 300 to be inserted under the skin of the patient using a minimally invasive procedure and to remain in the desired orientation during insert. For example, the IMD 300 may include a radial asymmetry (notably, a rectangular shape) along the longitudinal axis that maintains the device in the proper orientation following insertion. In one example, the spacing between the proximal electrode 302a and the distal electrode 302b may range from 30 millimeters (mm) to 55 mm, 35 mm to 55 mm, and from 40 mm to 55 mm and may be any range or individual spacing from 25 mm to 60 mm. In another example, the spacing between the proximal electrode 302a and the distal electrode 302b may range from 15 mm to 30 mm, 17 mm to 28 mm, and from 20 mm to 28 mm and may be any range or individual spacing from 12 mm to 30 mm. In addition, the length L of the IMD 300 may range from 30 mm to about 70 mm. In other embodiments, the length L may range from 40 mm to 60 mm, 45 mm to 60 mm and may be any length or range of lengths between about 30 mm and about 70 mm. In some examples, the length L of the IMD 300 may range from 15 mm to about 35 mm, or from 20 mm to 30 mm, 22 mm to 30 mm and may be any length or range of lengths between about 15 mm and about 35 mm. In addition, the width W of a major surface of the IMD 300, e.g., the insulative cover 316 in the example shown in FIG. 3, may range from 3 mm to 10 mm and may be any single or range of widths between 3 mm and 10 mm, or may range from 1.5 mm to 5 mm and may be any single or range of width between 1.5 mm and 5 mm. The thickness or depth D of the IMD 300 may range from 2 mm to 9 mm, or from 1.5 mm to 4.5 mm. In other embodiments, the depth D of the IMD 300 may range from 2 mm to 5 mm and may be any single or range of depths from 2 mm to 9 mm, or may range from 1 mm to 2.5 mm and may be any single or range of depths from 1 mm to 4.5 mm. In addition, the IMD 300, according to an example of the present technology, may have a geometry and size designed for ease of implant and patient comfort. Examples of the IMD 300 described in this disclosure may have a volume of 3 cubic cm or less, 1.5 cubic cm or less or any volume between 3 and 1.5 cubic cm, or may have a volume of 1.5 cubic cm or less, 0.75 cubic cm or less or any volume between 1.5 and 0.75 cubic cm.

The depiction of different features as modules in FIGS. 1-3 is intended to highlight different functional aspects and does not necessarily imply that such modules must be realized by separate hardware or software components. Rather, functionality associated with one or more modules may be performed by separate hardware, firmware and/or software components, or integrated within common hardware, firmware and/or software components.

Furthermore, it should be understood that the systems and methods described herein in accordance with the present technology are not limited to the implantable medical devices 200, 300 described herein with respect to FIGS. 2A-3. Rather, the systems and methods described herein in accordance with the present technology may additionally or alternatively be used in conjunction with other implantable medical devices, in conjunction with other cardiac monitoring devices (e.g., other leadless cardiac monitoring devices, cardiac monitoring devices with leads, etc.), and/or in conjunction with other optical sensing devices (e.g., a wearable device measuring PPG).

### II. Methods for Predicting Labor Onset

In some embodiments, the present technology provides methods for predicting the onset of labor in a pregnant patient using an implantable medical device (e.g., the medical device 108 of FIG. 1, the device 200 of FIGS. 2A and 2B, and/or the device 300 of FIG. 3). For example, an implantable medical device may include one or more sensors (e.g., an electrical sensor, an optical sensor, a motion sensor, a temperature sensor, an impedance sensor, an acoustic sensor, and/or a biochemical sensor) that is configured to generate sensor data of the patient, such as electrophysiological data (e.g., ECG/EGM data, echocardiogram data, electromyography data, impedance data), optical data (e.g., PPG data), motion data, posture data, temperature data, audio signal data, and/or biochemical signal data. The sensor data can be analyzed to determine one or more physiological characteristics of the patient that are relevant to predicting when the patient will undergo labor, such as heart rate (e.g., minimum heart rate, maximum rate, heart rate variability over time), blood pressure (e.g., systolic blood pressure, diastolic blood pressure), oxygen saturation level (e.g., an SpO2 indicative of blood oxygen saturation and/or an StO2 indicative of tissue oxygen saturation), respiration rate, fluid level (e.g., subcutaneous fluid level, amniotic fluid level), body temperature, activity level (e.g., resting, low activity, high activity), biochemical species level (e.g., blood glucose level, hormone level), and/or sleep parameters (e.g., total sleep per night, time spent in REM sleep per night, sleep position, night wakings).

The physiological characteristics can then be used to predict a likelihood of the patient experiencing labor onset during a future time period (e.g., at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 20 weeks, or 30 weeks in the future). In some embodiments, the prediction indicates the likelihood that the patient will experience premature labor onset in the future, e.g., the patient will enter labor before week 37 of the pregnancy and/or before an ideal due date for the patient.

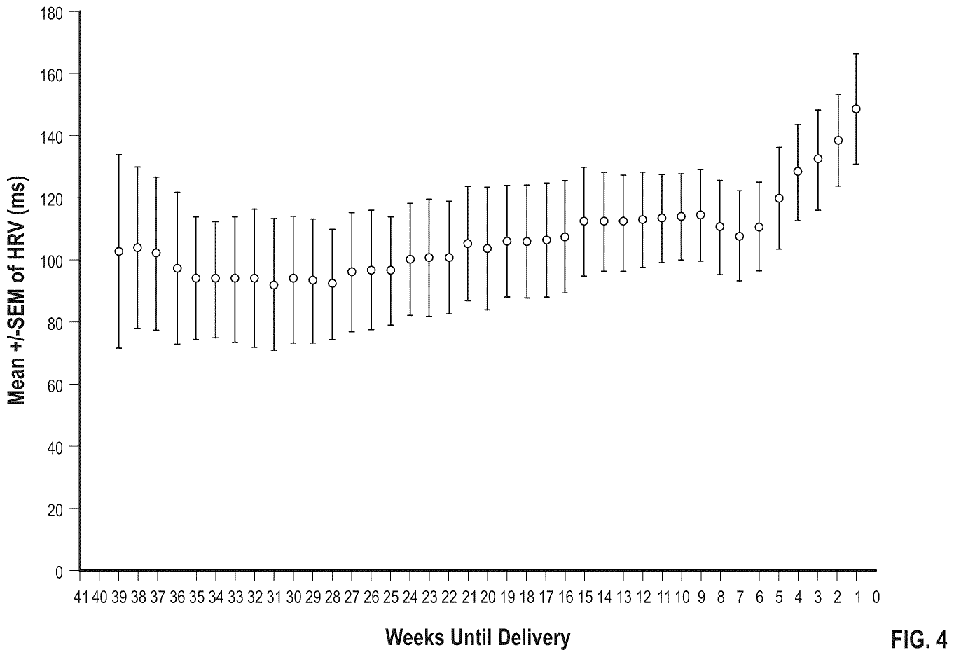

FIG. 4 is a graph illustrating an example relationship between heart rate variability (HRV) and time until delivery, in accordance with embodiments of the present technology. HRV data was collected using a Medtronic LINQ™ insertable cardiac monitor implanted in a pregnant patient over the course of 41 weeks leading up to the delivery date. HRV data was collected as a daily average from a group of pregnant patients and subsequently analyzed to extract information on the mean HRV and standard error of the mean (SEM) at weekly timepoints. As shown in FIG. 4, HRV demonstrates a significant upward trend over time starting at 7 weeks prior to the delivery date. This data indicates that HRV may be used to predict labor onset at least up to 7 weeks in advance.

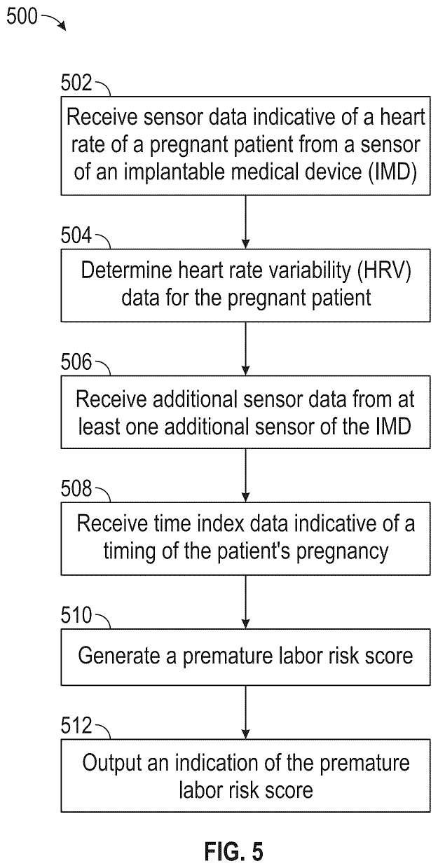

FIG. 5 is a flow diagram illustrating a method 500 for predicting premature labor onset in a pregnant patient, in accordance with embodiments of the present technology. The method 500 can be performed using any of the systems and devices described herein, such as the system 100 of FIG. 1, the device 200 of FIGS. 2A and 2B, and/or the device 300 of FIG. 3). In some embodiments, some or all of the processes of the method 500 are implemented as computer-readable instructions (e.g., program code) that are configured to be executed by one or more processors (e.g., processing circuitry 114 of the system 100 of FIG. 1).

The method 500 can begin at block 502 with receiving sensor data indicative of a heart rate of a pregnant patient. The sensor data can be received from a sensor of an implantable medical device, such as the sensor 110 of the medical device 108 of FIG. 1; the electrodes 204, 206 and/or the optical sensor arrangement 260 of the device 200 of FIGS. 2A and 2B; and/or the electrodes 302 and/or the optical sensor arrangement 310 of the device 300 of FIG. 3. The sensor can be an electrical sensor configured to measure electrophysiological characteristics indicative of heart rate (e.g., ECG data). For example, the implantable medical device may have a plurality of electrodes, such as a proximal electrode and a distal electrode located on the proximal and distal ends of an implantable medical device, respectively (e.g., as previously discussed with respect to FIGS. 2A-3). Alternatively, the electrode(s) may be positioned in a variety of other configurations on the implantable medical device. The electrode(s) may individually or collectively be used to sense electrical signals indicative of cardiac activity, which can then be processed to determine the heart rate of the patient. Alternatively or in addition, the sensor can be an optical sensor configured to measure optical signals indicative of heart rate (e.g., PPG data). For example, the implantable medical device may have an optical sensor arrangement including an emitter set of one or more optical light emitters and a detector set of one or more optical light detectors (e.g., as previously discussed with respect to FIGS. 2A-3). The optical sensor arrangement can, for example, be configured to provide a PPG signal using the emitter and detector sets, which can then be processed to determine the heart rate of the patient. In some embodiments, the heart rate may be determined based on two or more different types of sensor data (e.g., a heart rate based on ECG data, PPG data, and/or any of the other sensor data types described herein).

In some embodiments, the implantable medical device is an implantable cardiac monitor, as discussed in Section I above. For example, the implantable cardiac monitor may have a housing, a plurality of electrodes (e.g., a proximal electrode and distal electrode) configured to sense ECG signals, an optical sensor arrangement (e.g., one or more optical light emitters and one or more optical light detectors) configured to sense PPG signals, and electrical circuitry configured to receive and process the signals to determine cardiac activity of the patient (e.g., as previously discussed with respect to FIGS. 2A-3). The implantable cardiac monitor can be configured to be inserted under the skin of the patient proximate to the patient's heart using a minimally invasive procedure and to remain in the desired orientation after insertion. The implantable cardiac monitor may have a proximal end and a distal end that are rounded to reduce discomfort and irritation to surrounding tissue once inserted under the skin of the patient.

At block 504, the method 500 can include determining HRV data for the pregnant patient, based on the sensor data. HRV refers to the fluctuation in the time intervals between consecutive heartbeats (interbeat intervals) and may be determined by analyzing the patient's heart rate over a specified time period to calculate the magnitude of variations in the interbeat intervals over time. For example, the HRV data can be determined from ECG data collected from the patient, such as based on the R-R intervals, N-N intervals, and/or other interbeat interval metrics calculated from the ECG data. In some embodiments, the HRV data includes a mean HRV that is calculated by averaging the differences between interbeat intervals over a specified time period, e.g., using the root mean square of successive interbeat interval differences (RMSSD). Alternatively or in combination, HRV data can be determined through ensemble averaging of a set number of interbeat intervals that may be used as a rolling trend. HRV data can be adjusted and/or scaled based on heart rate during the period, or based on HRV interval only if gated by a set heart rate (e.g., low heart rate must be true before using HRV to prevent confounding factors, or HRV is bucketed within a set heart rate range for comparisons). Alternatively or in combination, the HRV data can include other statistics characterizing the differences between interbeat intervals, such as a standard deviation of the interbeat intervals (e.g., standard deviation of N-N intervals (SDNN), standard deviation of R-R intervals (SDRR), standard deviation of the average N-N intervals for each 5 minute segment of a 24 hour HRV recording (SDANN), mean of the standard deviations of all the N-N intervals for each 5 minute segment of a 24 hours HRV recording (SDNN index)), percentage of successive R-R intervals that differ by more than 50 milliseconds (pNN50), average difference between the highest and lowest heart rates during each respiratory cycle, integral of the density of the R-R interval histogram divided by its height (HRV triangular index), baseline width of the R-R interval histogram (TINN), etc.

In some embodiments, the HRV data includes time series data including a plurality of HRV values (e.g., mean HRV values) for a plurality of time points over a specified time period. For example, the HRV data may include a HRV value calculated once every minute, 2 minutes, 5 minutes, 10 minutes, 15 minutes, 20 minutes, 30 minutes, every hour, 5 hours, 10 hours, 12 hours, 24 hours, 36 hours, 48 hours, etc., over a specified time period of 12 hours, 24 hours, 36 hours, 48 hours, 3 days, 4 days, 5 days, 6 days, 1 week, 2 weeks, 3 weeks, 4 weeks, 8 weeks, 10 weeks, 12 weeks, 15 weeks, 20 weeks, 30 weeks, or 40 weeks, etc. Monitoring of HRV over time can reveal trends that can be used to predict labor onset. For example, as previously discussed with respect to FIG. 4, HRV may increase starting at 7 weeks prior to the expected delivery date.

Optionally, the HRV data can be determined in a manner to mitigate or avoid confounding factors that may potentially impact the corresponding HRV values. For instance, high-intensity activities such as exercise may lead to increased heart rates and/or HRV that may not be representative of the patient's baseline state. The effects of the confounding factors can be mitigated or avoided by measuring HRV from heart rate data obtained when the patient is expected to have a consistent heart rate, e.g., when the patient is at rest and/or sleeping (e.g., measuring during sleep or right after the patient wakes). For example, periods of rest and/or sleep can be detected based on physiological data of the patient (e.g., activity levels, respiration). Alternatively or in addition, the effects of confounding factors can be mitigated or avoided by measuring HRV with gating by a motion sensor (e.g., an accelerometer) or through adjusting, scaling, and/or bucketing the HRV measurement based on heart rate as discussed above.

At block 506, the method 500 can optionally include receiving additional sensor data from at least one additional sensor of the implantable medical device. For example, the additional sensor data can include atrial fibrillation (AF) burden, blood pressure (e.g., systolic blood pressure, diastolic blood pressure), oxygen saturation level (e.g., an SpO₂ indicative of blood oxygen saturation and/or an StO₂ indicative of tissue oxygen saturation), respiration rate, fluid level (e.g., subcutaneous fluid level, amniotic fluid level), body temperature, activity level (e.g., resting, low activity, high activity), biochemical species level (e.g., blood glucose level, hormone level), and/or sleep parameters (e.g., total sleep per night, time spent in REM sleep per night, sleep position, night wakings). The additional sensor(s) can be any of the sensor types described herein, such as electrical sensors (e.g., electrodes), optical sensors (e.g., photoplethysmography (PPG) sensors), motion sensors (e.g., accelerometers, gyroscopes, magnetometers, inertial measurement units (IMU)), temperature sensors (e.g., thermistors), impedance sensors, acoustic sensors (e.g., microphones), and/or biochemical sensors. The additional sensor data can be used to provide a more accurate and/or comprehensive prediction of labor onset, and/or may correct for factors that may confound HRV (e.g., exercise).

In some embodiments, the sensor data and/or the additional sensor data are received during a first time period. The first time period can be any suitable time period, such as a time period of at least 12 hours, 24 hours, 36 hours, 48 hours, 3 days, 4 days, 5 days, 6 days, 1 week, 2 weeks, 3 weeks, 4 weeks, 8 weeks, 10 weeks, 12 weeks, 15 weeks, 20 weeks, 30 weeks, or 40 weeks. The sensor data and/or the additional sensor data may be received at any suitable frequency during the first time period, such as once every minute, 2 minutes, 5 minutes, 10 minutes, 15 minutes, 20 minutes, 30 minutes, every hour, 2 hours, 5 hours, 10 hours, 12 hours, 24 hours, 36 hours, 48 hours, etc. The sensor data may be received at the same frequency as some or all of the additional sensor data, or the sensor data may be received at a different frequency than some or all of the additional sensor data.

At block 508, the method 500 can continue with receiving time index data indicative of a timing of the patient's pregnancy. For example, the time index data can include the patient's estimated date of conception and/or the patient's ideal delivery date (e.g., 37 to 40 weeks after the date of conception). The time index data can be received through user input (e.g., from the patient and/or the clinician). The time index data can be used as a reference point for where the patient currently is in the pregnancy timeline. Alternatively or in combination, the time index data can be used to determine whether the predicted timing of labor onset would be considered premature labor onset, e.g., labor occurring before week 37 of the patient's pregnancy and/or before an ideal due date for the patient may be classified as premature labor onset.

At block 510, the method 500 can include generating a premature labor risk score, based on the HRV data, additional sensor data (if present), and time index data. The premature labor risk score can represent a likelihood that the pregnant patient will experience labor onset at a future time, e.g., during a second time period subsequent to the first time period when the sensor data and/or additional sensor data was obtained. For example, the second time period can be a time period of at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 20 weeks, or 30 weeks after the first time period. In some embodiments, the second time period is up to 7 weeks after the first time period.

The premature labor risk score can be a quantitative score (e.g., numerical value, probability, percentage), a qualitative score (e.g., description, categorization), or any suitable combination thereof. For example, a premature labor risk score of 30% may represent a 30% likelihood that the patient will experience premature labor onset within the second time period (e.g., within the next X weeks). As another example, the premature labor risk score may be a value selected from a range from 0 to 10, where 0 represents no risk of premature labor onset within the second time period and 10 represents an extremely high risk of premature labor onset within the second time period. In a further example, the premature labor risk score may be a qualitative categorization, such as “high risk,” “intermediate risk,” “low risk.” As another example, the premature labor risk score may be a value indicating the number of weeks until labor onset, where greater risk of premature labor onset is identified when the number of weeks is below a certain threshold value.

In some embodiments, the premature labor risk score is generated using a machine learning algorithm. Examples of machine learning algorithms that may be used include any of the following: a regression algorithm (e.g., ordinary least squares regression, linear regression, logistic regression, stepwise regression, multivariate adaptive regression splines, locally estimated scatterplot smoothing), an instance-based algorithm (e.g., k-nearest neighbor, learning vector quantization, self-organizing map, locally weighted learning), regularization algorithms (e.g., ridge regression, least absolute shrinkage and selection operator, elastic net, least-angle regression), a decision tree algorithm (e.g., Iterative Dichotomiser 3 (ID3), C4.5, C5.0, classification and regression trees, chi-squared automatic interaction detection, decision stump, M5), a Bayesian algorithm (e.g., naïve Bayes, Gaussian naïve Bayes, multinomial naïve Bayes, averaged one-dependence estimators, Bayesian belief networks, Bayesian networks, hidden Markov models, conditional random fields), a clustering algorithm (e.g., k-means, single-linkage clustering, k-medians, expectation maximization, hierarchical clustering, fuzzy clustering, density-based spatial clustering of applications with noise (DBSCAN), ordering points to identify cluster structure (OPTICS), non negative matrix factorization (NMF), latent Dirichlet allocation (LDA), Gaussian mixture model (GMM)), an association rule learning algorithm (e.g., apriori algorithm, equivalent class transformation (Eclat) algorithm, frequent pattern (FP) growth), an artificial neural network algorithm (e.g., perceptrons, neural networks, back-propagation, Hopfield networks, autoencoders, Boltzmann machines, restricted Boltzmann machines, spiking neural nets, radial basis function networks), a deep learning algorithm (e.g., deep Boltzmann machines, deep belief networks, convolutional neural networks, stacked auto-encoders), a dimensionality reduction algorithm (e.g., principle component analysis (PCA), independent component analysis (ICA), principle component regression (PCR), partial least squares regression (PLSR), Sammon mapping, multidimensional scaling, projection pursuit, linear discriminant analysis, mixture discriminant analysis, quadratic discriminant analysis, flexible discriminant analysis), an ensemble algorithm (e.g., boosting, bootstrapped aggregation, AdaBoost, blending, gradient boosting machines, gradient boosted regression trees, random forest), or suitable combinations thereof.

In some embodiments, the machine learning algorithm is or includes a recurrent neural network (RNN). An RNN is a type of artificial neural network that may be used for sequential data processing, such as processing of time series data. For example, RNNs can process data across multiple time steps using recurrent units which have memory that is updated at each time step based on the current input and the previous state of the recurrent unit, thereby allowing the neural network to incorporate information from both of the previous and current inputs into the learning process. Examples of RNN architectures include long short term memory (LSTM), fully recurrent neural networks (FRNN), Hopfield networks, Elman networks, Jordan networks, gated recurrent units (GRU), bidirectional associative memory (BAM) networks, echo state networks (ESN), recursive neural networks, Neural Turing machines (NTM), etc.

The machine learning algorithm can be trained to predict the premature labor risk score from HRV data, additional sensor data (if present), and/or time index data. The input training data for the algorithm can include prior patient data from a plurality of pregnant patients, such as HRV data, additional sensor data, and/or labor onset data (e.g., when the patients actually experienced labor onset). The input training data can be grouped into individual training input vectors corresponding to different patients from the prior patient data. The output training vector for the algorithm can include actual labor onset date and/or the premature labor risk score associated with each corresponding training input vector. Each training input vector and associated training output vector can be grouped into a data pair to define the training data set for the machine learning algorithm. As the machine learning algorithm processes the training data set, the machine learning algorithm parameters may converge to learn correlations between input data and corresponding output data. Once trained with the training data set, the machine learning algorithm may be trained to receive a current input vector indicative of patient HRV data, additional sensor data, and/or time index data, and to determine an output vector indicative of labor onset date and/or generate a premature labor risk score by mapping the current input vector onto an output space, where the output space is defined at least in part by the plurality of training output vectors.

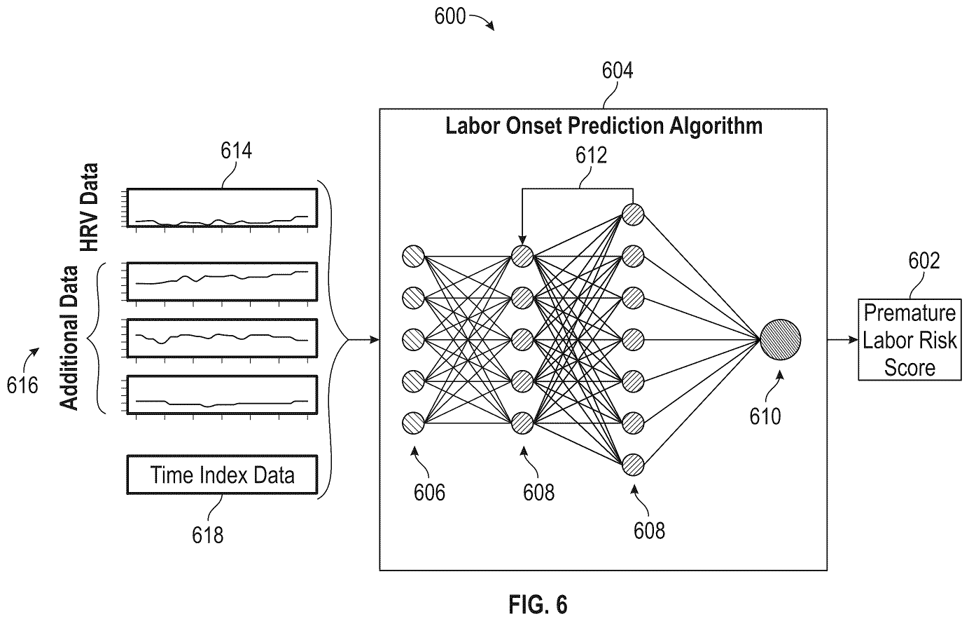

FIG. 6 is a block diagram illustrating an algorithm architecture 600 for generating a premature labor risk score 602, in accordance with embodiments of the present technology. The algorithm architecture 600 may be used in the process of block 510 of FIG. 5. As shown in FIG. 6, algorithm architecture 600 includes a labor onset prediction algorithm 604 that is configured to determine the premature labor risk score 602. In the illustrated embodiments, the labor onset prediction algorithm 604 is an RNN including an input layer 606, one or more hidden layers 608, and an output layer 610. The input layer 606, hidden layers 608, and output layer 610 can each include a plurality of interconnected nodes (also known as neurons), where the weights assigned to the connections between nodes are learned through training of the RNN (e.g., via backpropagation through time (BPTT)). Some or all of the hidden layers 608 may include recurrence (indicated by arrow 612), such that the nodes of the hidden layer 608 operate on data from one or more previous time steps in addition to data from the current time step.

The input layer 606 of the labor onset prediction algorithm 604 may receive one or more types of input data, including HRV data 614 (e.g., mean HRV over a first time period), additional data 616 (e.g., activity levels, sleep parameters, fluid levels, respiration rate, blood pressure, body temperature, biochemical species levels, etc., over the first time period, history of premature labor, risk factors related to premature labor), and/or time index data 618 (e.g., estimated conception date and/or estimated ideal due date). At least a portion of the input data may be time series data. For example, the HRV data 614 can include a plurality of HRV values (e.g., mean HRV) measured at a plurality of time points (e.g., daily, weekly) over the first time period. Similarly, the additional data 616 can include activity level data, sleep parameter data, fluid level data, respiration rate data, blood pressure data, body temperature data, biochemical species level data, etc., measured at a plurality of time points over the first time period. At least a portion of the input data may be non-time series data (e.g., individual numerical values, Boolean values, categorizations). For example, the time index data 618 can be a single value representing the estimated conception date and/or estimated ideal due date. Optionally, the additional data 616 can alternatively or additionally include non-time series data. For instance, the additional data 616 can include a Boolean value indicating whether the patient has a history of premature labor and/or whether any other risk factors for premature labor are present.

The input data (e.g., HRV data 614, additional data 616, and/or time index data 618) is input into the labor onset prediction algorithm 604, which then generates the premature labor risk score 602 as output. Optionally, the different types of input data may be weighted differently in determining the premature labor risk score 602, e.g., based on the reliability of the corresponding sensor, data quality, input from the clinician, preexisting conditions and/or other patient-specific factors, etc. The premature labor risk score 602 can be a quantitative score (e.g., numerical value, probability, percentage), a qualitative score (e.g., description, categorization), or any suitable combination thereof that represents the likelihood of the patient experiencing early labor onset at a second time period subsequent to the first time period.

In some embodiments, the labor onset prediction algorithm 604 is configured to produce other types of output data in addition to the premature labor risk score 602, such as a prediction of the labor onset date (e.g., the number of weeks before labor onset, which may be compared to a threshold to assess whether the predicted labor onset is premature), a prediction of a risk score for other types of conditions (e.g., a preeclampsia risk score), etc. In such embodiments, the output layer 610 can include a plurality of nodes, with each node corresponding to a different type of output data (e.g., a first node corresponding to the premature labor risk score 602, a second node corresponding to a preeclampsia risk score, etc.). Moreover, the training of the labor onset prediction algorithm 604 can be adjusted as appropriate to produce the additional output data, as discussed elsewhere herein.

Referring again to block 512, the method 500 can continue with outputting an indication of the premature labor risk score. The indication of the premature labor risk score can be in any suitable output format, such as numbers, text, graphs, visualizations, haptics, audio, etc. Optionally, the indication can include a plurality of premature labor risk scores calculated at different time points throughout the patient's pregnancy, e.g., to allow the patient and/or clinician to assess how the risk has changed over time. The indication can be transmitted to a computing device associated with the patient and/or the clinician (e.g., patient IO device 118 and clinician IO device 120, as previously discussed with respect to FIG. 1) or any other suitable device (e.g., external device 116, as previously discussed with respect to FIG. 1). The indication may include additional information in addition to the premature labor risk score to aid in pregnancy-related decisions, such as treatment recommendations (e.g., steroid injections, medications, cervical cerclage, bed rest, hydration, notifying the patient to be in closer proximity to a site of care).

Optionally, the process of block 512 can include outputting an indication of whether the patient is at high risk of experiencing premature labor. For example, the premature labor risk score may be compared to a threshold. If the premature labor risk score is determined to be greater than the threshold, then the patient may be considered at high risk of premature labor. If the premature labor risk score is determined to be less than the threshold, then the patient may not be considered at high risk of premature labor. The threshold may be a constant value or the value of the threshold may vary over time. For instance, the threshold may be adjusted based on the time index data to become more sensitive later in the patient's pregnancy.

The threshold can be a generic threshold (e.g., the value of the threshold is uniform across all patients or for a particular category of patients) or the threshold can be a patient-specific threshold (e.g., the value of the threshold may be individualized to each patient). In some embodiments, a patient-specific threshold is customizable based on patient-specific factors that may impact the patient's likelihood of experiencing premature labor, such as demographic characteristics (e.g., age, race, ethnicity), medical history (e.g., weight, preexisting and/or chronic conditions, previous preterm delivery, medications), social characteristics, behavioral characteristics, etc. For instance, the threshold may be adjusted for greater sensitivity (e.g., lowered) if the patient has certain preexisting conditions that increase the likelihood of adverse outcomes. Alternatively or in combination, the patient-specific threshold may be set based on input from the clinician, e.g., the clinician may decide whether the threshold for the patient should be more or less sensitive.

If the patient is determined to be at high risk of premature labor, an additional alert can be generated and output to the patient and/or clinician. The alert of the patient being at high risk of premature labor can be in any suitable output format, such as numbers, text, graphs, visualizations, haptics, audio, etc. The alert can be transmitted to a computing device associated with the patient and/or the clinician (e.g., patient IO device 118 and clinician IO device 120, as previously discussed with respect to FIG. 1) or any other suitable device (e.g., external device 116, as previously discussed with respect to FIG. 1). If the patient is determined to be high risk, the system may generate treatment recommendations (e.g., steroid injections) and/or output the recommendations with the alert.

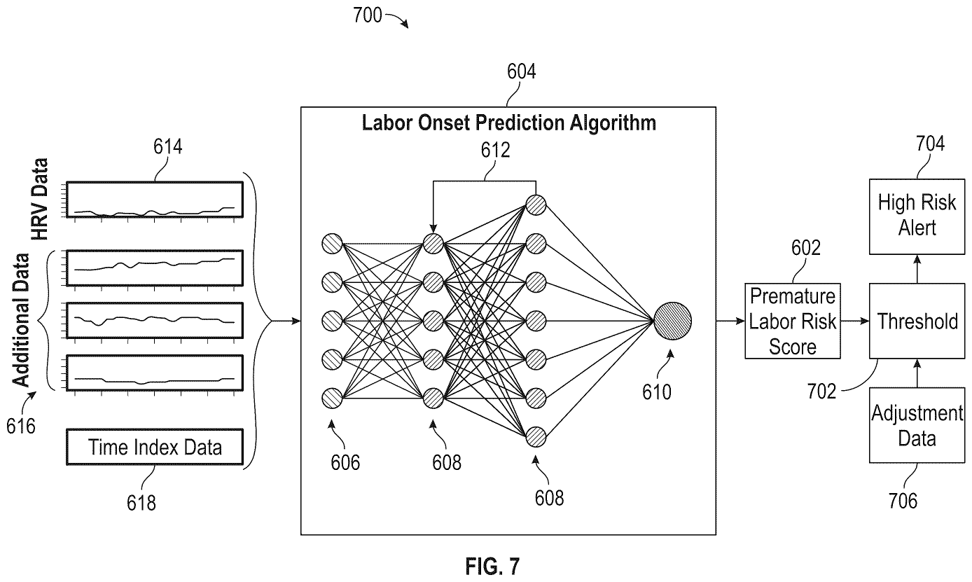

FIG. 7 is a block diagram illustrating an algorithm architecture 700 for determining whether a patient is at high risk of premature labor, in accordance with embodiments of the present technology. The algorithm architecture 700 may be used in the process of block 512 of FIG. 5. As shown in FIG. 7, the algorithm architecture 700 includes a labor onset prediction algorithm 604, that is configured to generate a premature labor risk score 602 based on HRV data 614, additional data 616, and/or time index data 618, e.g., as previously described with respect to FIG. 6. The premature labor risk score 602 can be compared to a threshold 702. If the premature labor risk score 602 is determined to be greater than the threshold 702, a high risk alert 704 can be generated to indicate that the patient is at high risk of experiencing premature labor. Optionally, adjustment data 706 may be used to modify the threshold 702, e.g., based on patient-specific factors that may impact the patient's likelihood of experiencing premature labor. The adjustment data 706 may include data indicative of the patient's demographic characteristics, medical history, social characteristics, behavioral characteristics, etc., input data from the clinician, and/or the time index data 618.

Although FIG. 7 illustrates the threshold 702 as being separate from the labor onset prediction algorithm 604, in other embodiments, the labor onset prediction algorithm 604 can be configured to determine the high risk alert 704 in combination with generating the premature labor risk score 602, such that the thresholding process is integrated into the labor onset prediction algorithm 604. In such embodiments, the adjustment data 706 can be provided as an additional input to the labor onset prediction algorithm 604, and the high risk alert 704 can be an additional output of the labor onset prediction algorithm 604. The training data from the labor onset prediction algorithm 604 can include demographic characteristics, medical history, social characteristics, behavioral characteristics, etc., for a plurality of previous patients so that the labor onset prediction algorithm 604 can learn the correlations between patient-specific factors and labor onset risk.

Referring again to FIG. 5, the method 500 can be modified in many different ways. For example, the ordering of the processes shown in FIG. 5 can be varied (e.g., the processes of blocks 504, 506, and/or 508 can be performed in any order). Some of the processes of the method 500 can be omitted (e.g., the process of block 506), and/or the method 500 can include additional processes not shown in FIG. 5. For example, the algorithms described herein (e.g., algorithm architectures 600, 700) may be modified to generate other outputs besides a premature labor risk score, such as a risk score representing a likelihood of one or more other obstetric conditions (e.g., preeclampsia, chronic hypertension, gestational hypertension, gestational diabetes, sepsis, cardiac arrhythmias, heart failure, hemorrhage, arteriovenous fistula, stroke, pre-partum or post-partum depression). In embodiments where optical sensor data is available, the method 500 can implement an algorithm that is configured to generate a preeclampsia risk score based on blood pressure data determined from the optical sensor data and/or the premature labor risk score. The algorithms described herein (e.g., algorithm architectures 600, 700) may be modified to accept other input data besides HRV data, additional sensor data, and time index data, such as data indicative of the patient's demographic characteristics (e.g., age, race, ethnicity, medical history (e.g., weight, preexisting and/or chronic conditions, previous preterm delivery, medications), social characteristics, behavioral characteristics, clinician input, etc. The weights of the input data may be adjusted based on the type of output to be produced by the algorithm, e.g., blood pressure data may be weighted more heavily if the algorithm is configured to predict preeclampsia.

### Examples

The following examples are included to further describe some aspects of the present technology, and should not be used to limit the scope of the technology.
Example 1. A system comprising:
   a medical device configured to be implanted in a pregnant patient, wherein the medical device comprises a sensor configured to obtain sensor data indicative of a heart rate of the pregnant patient over a first time period;
   processing circuitry; and
   a memory operably coupled to the processing circuitry and storing instructions that, when executed by the processing circuitry, cause the system to perform operations comprising:
      receiving the sensor data indicative of the heart rate of the pregnant patient over the first time period from the sensor,
      determining heart rate variability (HRV) data for the pregnant patient over the first time period based on the sensor data,
      receiving time index data indicative of a timing of the patient's pregnancy;
      generating a premature labor risk score based on the HRV data and the time index data, wherein the premature labor risk score represents a likelihood that the pregnant patient will experience premature labor onset during a second time period subsequent to the first time period, and
      outputting an indication of the premature labor risk score, wherein the indication is configured to be displayed to a user via a computing device.
Example 2. The system of Example 1, wherein the premature labor risk score is generated using a machine learning algorithm that is trained on HRV data and labor onset data from a plurality of pregnant patients.
Example 3. The system of Example 2, wherein the machine learning algorithm comprises a recurrent neural network.
Example 4. The system of any one of Examples 1 to 3, wherein the operations further comprise:
   comparing the premature labor risk score to a threshold value, and
   outputting an indication to the user that the pregnant patient is at high risk for premature labor onset in response to a determination that the premature labor risk score exceeds the threshold value.
Example 5. The system of Example 4, wherein the operations further comprise adjusting the threshold value based on a patient-specific characteristic, a user input, or a combination thereof.
Example 6. The system of any one of Examples 1 to 5, wherein the HRV data comprises time series data including a plurality of HRV values at a plurality of time points over the first time period.
Example 7. The system of any one of Examples 1 to 6, wherein the time index data comprises an estimated conception date, an estimated ideal due date, or a combination thereof.
Example 8. The system of any one of Examples 1 to 7, wherein the sensor comprises one or more electrodes and the sensor data comprises electrocardiogram (ECG) data.
Example 9. The system of any one of Examples 1 to 8, wherein the sensor comprises an optical sensor and the sensor data comprises photoplethysmography (PPG) data.
Example 10. The system of any one of Examples 1 to 9, wherein the medical device comprises at least one additional sensor configured to obtain additional sensor data of the pregnant patient over the first time period, and wherein the premature labor risk score is generated based on the additional sensor data.
Example 11. The system of Example 10, wherein the at least one additional sensor comprises one or more of the following: an optical sensor, a motion sensor, a temperature sensor, an impedance sensor, an acoustic sensor, or a biochemical sensor.
Example 12. The system of Example 10 or 11, wherein the additional sensor data is indicative of one or more of the following physiological characteristics: activity levels, sleep parameters, fluid levels, respiration rate, blood pressure, body temperature, or biochemical species levels.
Example 13. The system of any one of Examples 1 to 12, wherein the second time period is up to seven weeks after the first time period.
Example 14. The system of any one of Examples 1 to 13, wherein the processing circuitry and the memory are part of the medical device.
Example 15. The system of any one of Examples 1 to 13, wherein the processing circuitry and the memory are part of an external device separate from the medical device.
Example 16. The system of any one of Examples 1 to 15, wherein the medical device is an implantable cardiac monitor.
Example 17. A method comprising:
   receiving, via processing circuitry, sensor data indicative of a heart rate of a pregnant patient over a first time period, wherein the sensor data is received from a sensor of an implantable medical device;
   determining, via the processing circuitry, heart rate variability (HRV) data for the pregnant patient over the first time period based on the sensor data;
   receiving, via the processing circuitry, time index data indicative of a timing of the patient's pregnancy;
   generating, via the processing circuitry, a premature labor risk score based on the HRV data and the time index data, wherein the premature labor risk score represents a likelihood that the pregnant patient will experience premature labor onset during a second time period subsequent to the first time period, and
   outputting, via the processing circuitry, an indication of the premature labor risk score, wherein the indication is configured to be displayed to a user via a computing device.
Example 18. The method of Example 17, wherein generating the premature labor risk score comprises inputting the HRV data and the time index data into a machine learning algorithm that is trained on HRV data and labor onset data from a plurality of pregnant patients.
Example 19. The method of Example 18, wherein the machine learning algorithm comprises a recurrent neural network.
Example 20. The method of any one of Examples 17 to 19, further comprising:
   comparing, via the processing circuitry, the premature labor risk score to a threshold value, and
   outputting, via the processing circuitry, an indication to the user that the pregnant patient is at high risk for premature labor onset in response to a determination that the premature labor risk score exceeds the threshold value.
Example 21. The method of Example 20, further comprising adjusting the threshold value based on a patient-specific characteristic, a user input, or a combination thereof.
Example 22. The method of any one of Examples 17 to 21, wherein the HRV data comprises time series data including a plurality of HRV values at a plurality of time points over the first time period.
Example 23. The method of any one of Examples 17 to 22, wherein the time index data comprises an estimated conception date, an estimated ideal due date, or a combination thereof.
Example 24. The method of any one of Examples 17 to 23, wherein the sensor comprises one or more electrodes and the sensor data comprises electrocardiogram (ECG) data.
Example 25. The method of any one of Examples 17 to 24, wherein the sensor comprises an optical sensor and the sensor data comprises photoplethysmography (PPG) data.
Example 26. The method of any one of Examples 17 to 25, further comprising receiving, via the processing circuitry, additional sensor data of the pregnant patient over the first time period, wherein the premature labor risk score is generated based on the additional sensor data.
Example 27. The method of Example 26, wherein the additional sensor data is collected via one or more of the following: an optical sensor, a motion sensor, a temperature sensor, an impedance sensor, an acoustic sensor, or a biochemical sensor.
Example 28. The method of Example 26 or 27, wherein the additional sensor data is indicative of one or more of the following physiological characteristics: activity levels, sleep parameters, fluid levels, respiration rate, blood pressure, body temperature, or biochemical species levels.
Example 29. The method of any one of Examples 17 to 28, wherein the second time period is up to seven weeks after the first time period.
Example 30. The method of any one of Examples 17 to 29, wherein the processing circuitry is part of the implantable medical device.
Example 31. The method of any one of Examples 17 to 29, wherein the processing circuitry is part of an external device separate from the implantable medical device.
Example 32. The method of any one of Examples 17 to 31, wherein the implantable medical device is an implantable cardiac monitor.

### Conclusion

Although many of the embodiments are described above with respect to systems, devices, and methods for predicting premature labor onset, the technology is applicable to other applications and/or other approaches, such as predictions of other obstetric conditions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-7.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system comprising:
a medical device configured to be implanted in a pregnant patient, wherein the medical device comprises a sensor configured to obtain sensor data indicative of a heart rate of the pregnant patient over a first time period;
processing circuitry; and
a memory operably coupled to the processing circuitry and storing instructions that, when executed by the processing circuitry, cause the system to perform operations comprising:
receiving the sensor data indicative of the heart rate of the pregnant patient over the first time period from the sensor,
determining heart rate variability (HRV) data for the pregnant patient over the first time period based on the sensor data,
receiving time index data indicative of a timing of the patient's pregnancy;
generating a premature labor risk score based on the HRV data and the time index data, wherein the premature labor risk score represents a likelihood that the pregnant patient will experience premature labor onset during a second time period subsequent to the first time period, and
outputting an indication of the premature labor risk score, wherein the indication is configured to be displayed to a user via a computing device.

2. The system of claim 1, wherein the premature labor risk score is generated using a machine learning algorithm that is trained on HRV data and labor onset data from a plurality of pregnant patients.

3. The system of claim 2, wherein the machine learning algorithm comprises a recurrent neural network.

4. The system of claim 1, wherein the operations further comprise:
comparing the premature labor risk score to a threshold value, and
outputting an indication to the user that the pregnant patient is at high risk for premature labor onset in response to a determination that the premature labor risk score exceeds the threshold value.

5. The system of claim 4, wherein the operations further comprise adjusting the threshold value based on a patient-specific characteristic, a user input, or a combination thereof.

6. The system of claim 1, wherein the HRV data comprises time series data including a plurality of HRV values at a plurality of time points over the first time period.

7. The system of claim 1, wherein the time index data comprises an estimated conception date, an estimated ideal due date, or a combination thereof.

8. The system of claim 1, wherein the sensor comprises one or more electrodes and the sensor data comprises electrocardiogram (ECG) data.

9. The system of claim 1, wherein the sensor comprises an optical sensor and the sensor data comprises photoplethysmography (PPG) data.

10. The system of claim 1, wherein the medical device comprises at least one additional sensor configured to obtain additional sensor data of the pregnant patient over the first time period, and wherein the premature labor risk score is generated based on the additional sensor data.

11. The system of claim 10, wherein the at least one additional sensor comprises one or more of the following: an optical sensor, a motion sensor, a temperature sensor, an impedance sensor, an acoustic sensor, or a biochemical sensor.

12. The system of claim 10, wherein the additional sensor data is indicative of one or more of the following physiological characteristics: activity levels, sleep parameters, fluid levels, respiration rate, blood pressure, body temperature, or biochemical species levels.

13. The system of claim 1, wherein the premature labor risk score is generated based on additional data, wherein the additional data is indicative of whether the pregnant patient has one or more risk factors for premature labor onset.

14. The system of claim 13, wherein the one or more risk factors comprise a history of premature labor onset.

15. The system of claim 1, wherein the second time period is up to seven weeks after the first time period.

16. The system of claim 1, wherein the processing circuitry and the memory are part of the medical device.

17. The system of claim 1, wherein the processing circuitry and the memory are part of an external device separate from the medical device.

18. The system of claim 1, wherein the medical device is an implantable cardiac monitor.

19. The system of claim 1, wherein the operations further comprise a generating risk score representing a likelihood of one or more other obstetric conditions.

20. The system of claim 19, wherein the one or more other obstetric conditions comprises one or more of the following: preeclampsia, chronic hypertension, gestational hypertension, gestational diabetes, sepsis, cardiac arrhythmias, heart failure, hemorrhage, arteriovenous fistula, stroke, pre-partum depression, or post-partum depression.
